(19) United States
(12) Patent Application Publication
(10) Pub. No.: US 2006/0153812 A1
(43) Pub. Date: Jul. 13, 2006

(54) Medium for stem cells to be used in intervertebral disk generation and regeneration of intervertebral disk using stem cells

(76) Inventors: Joji Mochida, Kanagawa (JP); Daisuke Sakai, Kanagawa (JP); Takeshi Nomura, Kanagawa (JP); Kazuhiro Nishimura, Kanagawa (JP); Masahiko Okuma, Kanagawa (JP); Yukihiro Yamamoto, Kanagawa (JP)

Correspondence Address: ARENT FOX PLLC, 1050 CONNECTICUT AVENUE, N.W., SUITE 400, WASHINGTON, DC, 20036, US

(21) Appl. No.: 10/546,049

(22) PCT Filed: Feb. 25, 2003
(86) PCT No.: PCT/JP03/02065

§ 371 Date: Aug. 17, 2005

(51) Int. Cl. A61K 35/30; C12N 5/08

(52) U.S. Cl. 424/93.7; 435/368; 435/404

(57) ABSTRACT

A medium for stem cell in regeneration of intervertebral disc comprising an autologous serum obtained by sterilization and immobilization of serum of an individual having the intervertebral disc to be regenerated, a medium for cell culture and at least one antibiotic and a method for regenerating an intervertebral disc comprising suspending, in the above-mentioned medium or embedding in a cell carrier, stem cells collected from the subject individual or a homogenous or heterogenous individual, either directly or after culturing the same using the medium for regenerating the stem cell for regenerating intervertebral disc; and then transplanting the suspension or the embedded carrier of the stem cells into the nucleus pulposus cavity of the intervertebral disc.

### TECHNICAL FIELD

The present invention relates to a medium for stem cell in regeneration of intervertebral discs and treatment of low back pain, especially disorders of intervertebral disc accompanied by the intervertebral disc degeneration.

### BACKGROUND ART

Low back pain accounts for over 40% of patients presenting orthopaedic clinics. It is a problem which arose when human beings, who originally walked on all fours, started standing up. As the main cause of low back pain, the disorders of the intervertebral disc accompanied by intervertebral disc degeneration may be mentioned. Among these, intervertebral disc herniation often occurs in the young and adult ages of the 20s to 40s and cause social problems such as restrictions in daily life and obstruction of works. As a method for treatment of intervertebral disc herniation, posterior herniotomy is the general practice at present. However, this treatment results, after the surgery, in a cavity inside the intervertebral disc after the nucleus pulposus is extracted or delivered from the intervertebral disc. Since the nucleus pulposus essential for maintenance of the intervertebral discs is lost, there is the problem that the intervertebral disc steadily degenerates and reoccurrence of low back pain is promoted. Further, in non-surgically treated intervertebral disc herniation or lumbar spondylolisthesis or lumbar spondylosis deformans as well, the progress of intervertebral disc degeneration triggers the occurrence of low back pain.

However, at the present time, almost no clinically effective means is recognized for regeneration of intervertebral discs.

The present inventors previously experimentally proved that it is possible to suppress degeneration of intervertebral disc tissue by the reinserting nucleus pulposus cells reactivated by their unique coculture system into the nucleus pulposus cavity of intervertebral discs (see the following Non-patent Documents 1 to 3) and started clinical applications thereof in 2000.

Non-patent Document 1: Nishimura, K., Mochida, J.: Percutaneous reinsertion of the nucleus pulposus—An experimental study—, Spine 22: 1531-1539; 1998

Non-patent Document 2: Okuma, M., Mochida, J., Nishimura, K., Sakabe, K., Seiki, K.: Reinsertion of activated nucleus pulposus cells retards intervertebral disc degeneration—An in vitro and in vivo experimental study—. J. Orthop Res. 18: 983-997; 2000

Non-patent Document 3: Nomura, T., Mochida, J., et al.: Nucleus pulposus allograft retards intervertebral disc degeneration—An in vivo experimental study—, Clin. Orthop., 388: 94-101; 2001

However, our these techniques have the problem that broad clinical applications are not practical, since there are few cells which can be collected from humans and that the only way to obtain fresh nucleus pulposus is to collect nucleus pulposus from healthy intervertebral discs.

### DISCLOSURE OF THE INVENTION

Accordingly, the objects of the present invention are to overcome the conventional problems in the treatment of disorders of intervertebral disc explained above and to provide a method of regeneration of an intervertebral disc capable of being broadly and generally applied to disorders accompanied by intervertebral disc degeneration and a medium for stem cell usable for the same.

In accordance with the present invention, there is provided a medium for stem cell in regeneration of intervertebral disc comprising an autologous serum obtained by sterilization and immobilization of serum of an individual having the intervertebral disc to be regenerated, a medium for cell culture and at least one antibiotic.

In accordance with the present invention, there is also provided a method for regenerating an intervertebral disc comprising:

suspending, in the medium or embedding in a cell carrier, stem cells collected from the subject individual or a homogenous or heterogenous individual, either directly or after culturing the same using a cell culturing technology, especially using the culture for the stem cell in regeneration of intervertebral disc of the present invention; and then

transplanting the suspension or the embedded carrier of the stem cells into the nucleus pulposus cavity of the intervertebral disc.

### BRIEF DESCRIPTION OF THE DRAWINGS

The present invention will now be explained in detail with reference to the drawings:

FIG. 1(a) is a drawing (photograph) showing the normal state of an intervertebral disc of a rabbit used in the Examples, FIG. 1(b) is a drawing (photograph) showing the degenerated state and FIG. 1(c) is a drawing (photograph) showing a sagittal-section of the intervertebral disc in the state after regeneration,

FIG. 2(a) is a drawing (photograph) showing the normal state of an intervertebral disc of a rabbit used in the Examples, FIG. 2(b) is a drawing (photograph) showing the degenerated state, and FIG. 2(c) is a drawing (photograph) showing the tissue under a microscope in the sagittal direction of the intervertebral disc in the state after regeneration.

### DETAILED DESCRIPTION OF THE INVENTION

The main cause of chronic low back pain, that is, intervertebral disc degeneration, is an irreversible change. Since 1993, the present inventors have been experimentally proving the effect of reinsertion of activated nucleus pulposus in the suppression of intervertebral disc degeneration for the purpose of suppressing the intervertebral disc degeneration and regenerating intervertebral discs and have begun the clinical application thereof. However, there is a limit to the number of cells which can be collected from degenerated intervertebral discs. The only option is to collect fresh nucleus pulposus from healthy intervertebral discs, but the collection of fresh nucleus pulposus has been considered to be difficult in practice. Therefore, the present inventors attempted to regenerate intervertebral discs using mesenchymal stem cells, or multipotent stem cells having complete plasticity, and confirmed a certain effect in animal experiments, whereby the present invention has been completed.

The present inventors found that the mesenchymal stem cells or multipotent stem cells having complete plasticity transplanted into a degenerated intervertebral disc are later induced to intervertebral disc-like or intervertebral disc cells themselves, whereby the intervertebral discs are regenerated in function. This method, compared with our previously practiced method of reinsertion of autologous or allogenic intervertebral disc cells, does not require cells from healthy intervertebral discs and enables transplant cells to be relatively easily obtained and further is high in regenerative effect, and therefore, can be said to be an epoch-making method of regeneration of degenerated intervertebral discs, which is very well suited to clinical applications.

The present inventors do not know of any documentary reports on research relating to the regeneration of intervertebral discs using stem cells. Particularly, in the area of transplant into intervertebral discs, our report before the 17th Annual Research Meeting of the Japanese Orthopaedic Association (October 2002) is believed to have been the world's first report. When it becomes possible for this technique to be broadly clinically applied in the future, the present inventors believe the impact not only in medicine but also in society will be great, since there is currently no means effective for the suppression and treatment of intervertebral disc degeneration. This method is relatively easy to the clinical application due to the structure of intervertebral discs. Further, there has been experience in clinical application of nucleus pulposus reinsertion. Therefore, the present invention is expected to greatly contribute to society in both medical and economical viewpoints.

According to the present invention, the stem cells transplanted into the nucleus pulposus cavity of intervertebral disc provide tropic factors to the surrounding cells in the transplanted portion when transplanted. Further, the stem cells per se are induced to differentiate due to the tropic factors derived from the surrounding tissue and differentiation inducing factors etc. If these stem cells are transplanted into a degenerated intervertebral disc, they are induced to cells exhibiting the morphology of intervertebral disc cells and can regenerate intervertebral disc tissue. As such stem cells, it is possible to use those derived from the subject individual or homogenous or heterogenous individuals. Specifically, mesenchymal stem cells, multipotent stem cells, etc. may be mentioned. The collected stem cells may be directly suspended into our developed medium for stem cells of intervertebral disc regeneration or embedded in a cell carrier (for example, agarose, alginate, atelocollagen, etc.) for transplant. However, according to our experiments, the method of culturing and causing to proliferate the cells in our developed medium for stem cells in regeneration of intervertebral disc, then suspending them in the above medium or embedding them in a cell carrier and transplanting them into the nucleus pulposus cavity of intervertebral discs is preferable in that it has a high effect of regeneration of intervertebral discs.

As the medium for stem cells for intervertebral disc regeneration according to the present invention, it is possible to use a commercially available culture solution used in general for culturing mesenchymal stem cells, that is, a stem cell medium into which previously synthesized growth factors and serum derived from other animals are mixed. However, we conducted experiments using the following medium from the viewpoint that it is desirable to transplant cells in such a state as close as possible to the biological material receiving the transplant when transplanting stem cells in vivo and succeeded in causing the regeneration of intervertebral discs by transplanting mesenchymal stem cells into the nucleus pulposus cavity of intervertebral discs.

That is, first, we collected the necessary amount of whole blood from the individual for use in the transplant and used a centrifuge to separate the blood cells to thereby obtain the serum. We sterilized this serum using a sterilization filter and, followed by heating in a thermostatic tank at, for example, 50 to 70° C., preferably 55 to 60° C., for, for example, 20 to 40 minutes, preferably 25 to 35 minutes, to immobilize it. Then, we injected this into a previously sterilized medium for cell culture, for example, DMEM (Dulbecco's Modified Eagle Medium), DMEM/F-12MEM (Minimum Essential Medium), RPMI1640, BME (Basal Medium Eagle), Brinster's BMOC-3, BGJb, CMRL 1066, F-10, F-12, Glasgow MEM, IMDM (Iscove's Dulbecco's Medium), McCoy's 5A Medium, MCDB131 Medium, Medium 199, NCTC-109 Medium, Waymouth's MB 752-1Medium, William's Medium E, Opti-MEM I Reduced-Serum Medium, or another cell culture medium so that said autologous plasma became a concentration of 1 to 25% by weight, preferably 5 to 20% by weight. Note that these media have been known from the past, as cell culture media, and are commercially available. These media can be used alone or in any mixture thereof.

According to the present invention, further, an antibiotic may be added to the medium in such a concentration that an antibacterial action can be provided and that the cultured cells can be survived, specifically for example, penicillin in 8,000 to 10,000 U/ml, streptomycin in 8,000 to 10,000 μg/ml, amphotericin B in 20 to 25 μg/ml, gentamycin in 0.5 to 50 μg/ml, hygromycin B in 25 to 1000 μg/ml, kanamycin sulfate in 0.5 to 50 μg/ml, actinomycin D in 0.5 to 50 μg/ml, neomycin sulfate in 8,000 to 10,000 μg/ml, etc. alone or in any mixture thereof so as to obtain the medium for stem cell in the regeneration of intervertebral disc.

The method of transplant of the stem cells into the intervertebral disc tissue is not particularly limited, but may be carried out by exposing the intervertebral disc, then using a syringe or another suitable means capable of injecting a liquid or gel-like cell carrier or, when using a scaffold (e.g., a bioabsorptive polymer etc.), directly placing the same in the intervertebral disc. In the case of human subjects, sometimes the intervertebral disc is cracked or has holes. In this case, to prevent leakage of the transplanted cells, a known conventional adhesive for biological tissue (e.g., fibrin) or periorsteum or another connective tissue is used for repair. This transplant method is used for the purpose of suppressing degeneration of intervertebral disc or regenerating an intervertebral disc at the time of surgery having a direct or indirect effect on the intervertebral disc (e.g., intervertebral disc herniotomy, etc.) Further, for non-surgically treated intervertebral disc herniation, lumbar spondylolisthesis and lumbar spondylosis deformans as well, when intervertebral disc degeneration is progressed, it can be suppressed or the disc regenerated by use of the present medium for stem cell in intervertebral disc regeneration and intervertebral disc regeneration method. In these cases, it is desirable that the transplanted stem cells used be autogenous, but cells obtained from homogenous or heterogenous individuals may also be used.

### EXAMPLES

Examples of the method of transplanting stem cells into an intervertebral disc will now be explained in detail, but the present invention is by no means limited to the following Examples.

Preparation of Stem Cell Medium for Intervertebral Disc Regeneration

First, the necessary amount of whole blood was collected from the ear artery of each of the 12 week aged rabbits used for the transplants, then a centrifuge was used to separate the blood cells and obtain the serum. This serum was sterilized using a sterilization filter and was heated in a thermostatic chamber at 56° C. for 30 minutes to be immobilized. Next, the above autoserum was injected into DMEM (Dulbecco's Modified Eagle Medium) medium sterilized in advance to a concentration of 10%, then penicillin was added, as antibiotics, in an amount of 10,000 U/ml, streptomycin in an amount of 10,000 μg/ml and amphotericin B in an amount of 25 μg/ml to obtain a stem cell medium for intervertebral disc regeneration.

Preparation of Mesenchymal Stem Cells

The bone marrow fluid of each of 12 week aged rabbits was collected and a specific gravity separation solution was used to recover the monocyte layer. The monocyte layer thus recovered was inoculated in a culture flask and cultured in a 37° C. and 5% CO₂ atmosphere thermostatic chamber, until cells proliferated over substantially the entire surface of the flask (until reaching subconfluent) (about 14 days). Note that the medium was marked by introducing into the deposited cells proliferated by said stem cell medium for intervertebral disc regeneration an adeno or retrovirus vector having a LacZ (β-galactosidase) or GFP (Green Fluorescent Protein) marker gene incorporated thereinto so as to enable confirmation of the survival and activity after transplant. Thereafter, trypsin treatment was used to recover the cells for use as the transplant cells. The bone marrow mesenchymal stem cells thus obtained were used to induce cartilage, bone, and adipose cells. It was confirmed from the reproducibility of the plasticity that they were mesenchymal stem cells. Note that, in the case of human subjects, this can be confirmed by investigating the leukocyte surface antigens of bone marrow mesenchymal cells by flowcytometry.

Preparation of Rabbit Degenerated Intervertebral Discs

The 14 week aged same rabbits (after the elapse of two weeks) as used above were anesthesized, the four L2/L3 to L5/L6 intervertebral spaces were developed by an anterolateral approach, the intervertebral discs were pierced using 21 gauge needle syringes, and about 5 mg of the nucleus pulposus was aspirated so as to induce degeneration. Note that the suitability of this method for the preparation of an intervertebral disc degeneration model in this method is explained in the above Non-patent Documents 1 to 3 and other documents.

Transplant Technique

The mesenchymal stem cells obtained above were suspended in the above-mentioned intervertebral disc regeneration medium, then the cell suspension was directly, or after being embedded in the cell carrier Atelocollagen, transplanted to the degeneration-induced intervertebral discs of rabbits using a 27 gauge needle syringe.

Method of Evaluation

After the elapse of 2, 4, 8, 18, 24 and 48 weeks, MRI (Magnetic Resonance Imaging) was used to determine the water content in the intervertebral discs, then the rabbits were euthanized, the four treated intervertebral spaces were excized, fixed in formalin, decalcified and embedded in paraffin to prepare 4 μm thick sections in the sagittal direction. These sections were used for histological and immunohistological studies. The degree of degeneration and regeneration in the intervertebral disc tissue were evaluated.

Results

As a result of the above evaluation, in the group having transplanted mesenchymal stem cells according to the present invention, a relatively high value of the water content in the intervertebral disc as found by MRI was maintained, as compared with the control degenerated group having no transplant, and no reduction in the heights of the intervertebral discs as recognized in the control degenerated group could be observed. As a result of a histological study, the intervertebral disc cavity in which the mesenchymal stem cells were transplanted, were filled with intervertebral disc cell-like cells. Further, in the following five-rank evaluation of the classification of the degree of intervertebral disc degeneration, compared with the already most advanced degenerated Grade 4 to 5 in the 20 week aged (after elapse of eight weeks) control degenerated group, the ranking was the closest to normal Grade 0 to 1 in the group with stem cells transplanted even at 60 weeks age (after the elapse of 48 weeks). Further, in the proteoglycan staining intensity as well, the group having stem cells of the present invention transplanted exhibited a stainability of 3 to 3.5 times that of the control degenerated group, i.e., degeneration was excellently suppressed.

Grade 0: Normal

Grade 1: Mildly serpentine

Grade 2: Moderately serpentine

Grade 3: Severely serpentine with mild reversal

Grade 4: Severe reversal

Grade 5: Disappearance of annulus fibrosus

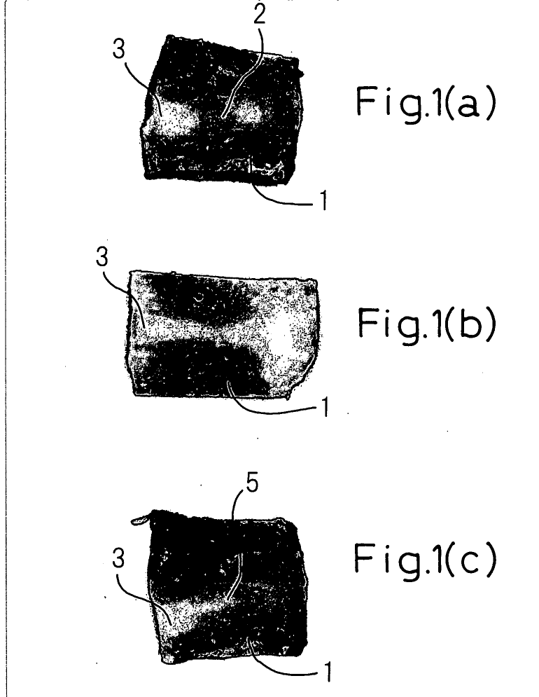

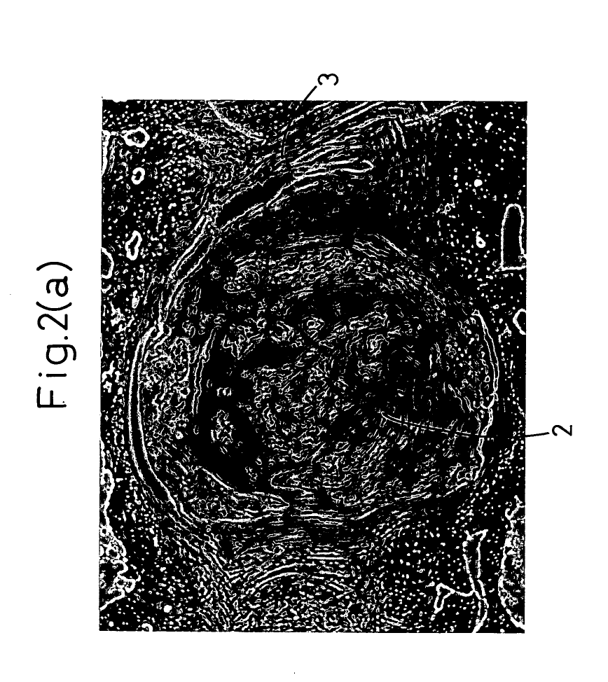

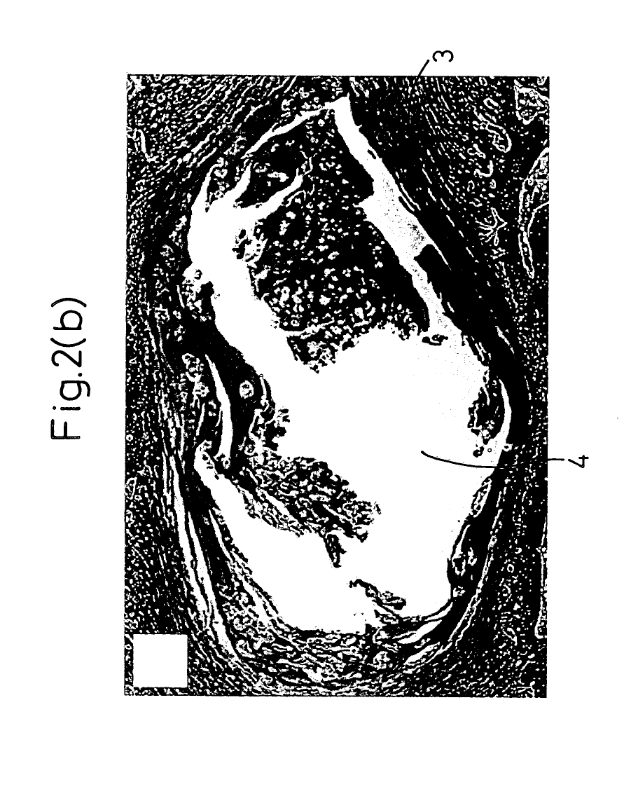

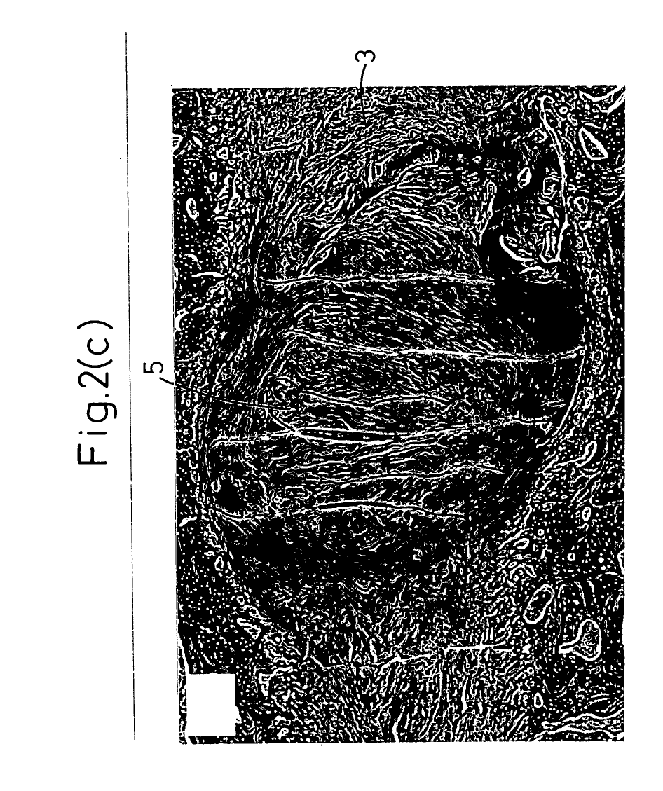

Photographs of the state of the intervertebral discs of rabbits used in the above examples are shown in FIGS. 1(a), (b), and (c) (magnification: 4) and FIGS. 2(a), (b), and (c) (magnification: 10). In these photographs, (a) shows the normal state, (b) the degenerated state, and (c) the regenerated state. FIGS. 1(a), (b), and (c) show the images of intervertebral discs as seen macroscopically along the sagittal direction, while FIGS. 2(a), (b), and (c) show tissues of sagittal-sections. In FIGS. 1(a), (b), and (c) and/or FIGS. 2(a), (b), and (c), 1 indicates a bone part, 2 the nucleus pulposus, 3 an annulus fibrosus, 4 a cavity, and 5 the transplanted stem cells. In the normal state (a), the nucleus pulposus 2 and annulus fibrosus 3 are present between the bone parts 1, and no degeneration of the intervertebral disc can be recognized, but in the degenerated state (b), there is no nucleus pulposus 2 and the height of the part of the annulus fibrosus 3 is reduced. In the state (c) after regeneration of this (after the elapse of 24 weeks), the transplanted stem cells 5 are induced to differentiate and, while different in morphology, form a state corresponding to the normal state (a).

### INDUSTRIAL APPLICABILITY

As explained above, by using the medium for stem cell for intervertebral disc regeneration according to the present invention, when transplanting mesenchymal stem cells into an intervertebral disc, degeneration of the intervertebral disc can be suppressed. Further, the transplanted stem cells are induced to differentiate to intervertebral disc-like cells by the growth factors and inducing factors etc. secreted from the transplant location, whereby the intervertebral disc can be regenerated. In this way, the mesenchymal stem cells are useful as new transplant materials for intervertebral disc regeneration. In particular, since the transplant of autologous bone marrow mesenchymal stem cells into the intervertebral disc is performed using completely autologous materials, it is considered a technique with immediate, great promise in clinical application. Note that a similar method may be used to regenerate an intervertebral disc using stem cells other than mesenchymal stem cells, for example, multipotent stem cells.

1. A medium for stem cell in regeneration of intervertebral disc comprising an autologous serum obtained by sterilization and immobilization of serum of an individual having the intervertebral disc to be regenerated, a medium for cell culture and at least one antibiotic.

2. A medium for stem cell as claimed in claim 1, wherein the concentration of the autologous serum in the medium for cell culture is 1 to 25% by weight and the concentration of the antibiotic in the medium is such a concentration that an antibacterial action is obtained and the cultured cells are able to be survived.

3. A medium for stem cell as claimed in claim 1, wherein said medium for cell culture is at least one medium selected from the group consisting of DMEM (Dulbecco's Modified Eagle Medium), DMEM/F-12MEM (Minimum Essential Medium), RPMI1640, BME (Basal Medium Eagle), Brinster's BMOC-3, BGJb, CMRL 1066, F-10, F-12, Glasgow MEM, IMDM (Iscove's Dulbecco's Medium), McCoy's 5A Medium, MCDB131 Medium, Medium 199, NCTC-109 Medium, Waymouth's MB 752-1Medium, William's Medium E and Opti-MEMI Reduced-Serum Medium.

4. A method for regenerating an intervertebral disc comprising:
suspending, in the medium according to claim 1 or embedding in a cell carrier, stem cells collected from the subject individual or a homogenous or heterogenous individual, either directly or after culturing the same using the medium for the stem cell according to claim 1; and then
transplanting the suspension or the embedded carrier of the stem cells into the nucleus pulposus cavity of the intervertebral disc.

5. A method as claimed in claim 4, wherein said stem cells are mesenchymal stem cells or multipotent stem cells.
